(19) United States
(12) Patent Application Publication
(10) Pub. No.: US 2024/0239726 A1
(43) Pub. Date: Jul. 18, 2024

(54) HIGHLY STRUCTURED, HIGH VINYLIDENE PROPYLENE OLIGOMER AND METHOD OF MAKING

(71) Applicant: CHEVRON U.S.A. INC., San Ramon, CA (US)

(72) Inventor: Sven Ivar HOMMELTOFT, Pleasant Hill, CA (US)

(21) Appl. No.: 18/576,716

(22) PCT Filed: May 16, 2022
(86) PCT No.: PCT/US2022/029364

§ 371 Date: Jan. 4, 2024

Related U.S. Application Data
(60) Provisional application No. 63/189,585, filed on May 17, 2021.

(51) Int. Cl. C07C 2/34; C07C 11/06

(52) U.S. Cl. CPC C07C 2/34; C07C 11/06; C07C 2531/14; C07C 2531/22

(57) ABSTRACT

A method for making a propylene oligomer having a high vinylidene content and a high degree of structural regularity and the propylene oligomer made therefrom. The method of making the propylene oligomer comprises contacting an unbridged metallocene complex with a propylene composition in the presence of a modified methyl aluminum oxide activator compound under suitable oligomerization conditions. The use of the combination of a modified methyl aluminum oxide activator compound with an unbridged metallocene complex has been found to provide increased vinylidene group content and reduced hydrogenated propylene oligomer content.

### CROSS-REFERENCE TO RELATED APPLICATIONS

This application claims the benefit of priority to U.S. Provisional Appl. Ser. No. 63/189,585, filed on May 17, 2021, entitled “HIGHLY STRUCTURED, HIGH VINYLIDENE PROPYLENE OLIGOMER AND METHOD OF MAKING”, the disclosure of which is incorporated herein by reference in its entirety.

### FIELD OF THE INVENTION

A method for making a propylene oligomer having a high vinylidene content and a high degree of structural regularity and the propylene oligomer made therefrom.

### BACKGROUND OF THE INVENTION

A variety of methods for making propylene oligomers are known in the art. As described in, e.g., U.S. Pat. Nos. 9,732,300 and 8,536,391, propylene oligomers may be made by the oligomerization of propylene feedstocks using a metallocene-based catalyst system. Useful metallocene catalyst systems include a wide variety of compounds comprising one or more transition metals from IUPAC Groups 3-12 of the Periodic Table of the Elements. More specific metallocene catalyst systems useful for propylene oligomerization include compounds wherein the transition metal is chromium, titanium, zirconium, or hafnium, comprising cyclopentadienyl substitute groups, either in bridged or unbridged form. In some cases, specific activators, such as aluminoxanes and/or chemically treated solid oxides have been used in conjunction with metallocene catalysts to produce propylene oligomers having beneficial properties.

Despite the diversity and complexity of metallocene catalyst system development, however, there remains a need for further and continuing efforts to expand the range of products produced using such catalysts. One such area concerns the preparation of functionalized oligomers, specifically propylene oligomers comprising a high content of terminal vinylidene groups. The present invention is directed to such needs and provides an advantageous solution.

### SUMMARY OF THE INVENTION

This invention relates to a method for making a propylene oligomer and to the propylene oligomer made therefrom. The method and propylene oligomer product provide certain advantages as compared with known methods of making propylene oligomers. For example, the propylene oligomer contains an increased vinylidene content as compared with propylene oligomers prepared by known methods. The propylene oligomer may also advantageously contain a reduced amount of hydrogenated propylene oligomer as compared with other methods of making propylene oligomers, e.g., using metallocene catalysts.

The propylene oligomer generally has a number average molecular weight Mₙ of about 200 to about 10,000, a vinylidene content of greater than about 70 mol %, and comprises less than about 6 wt. % of hydrogenated propylene oligomer. A propylene oligomer composition made according to the method generally comprises from about 70 wt. % to 100 wt. % of the propylene oligomer.

The method of making the propylene oligomer comprises contacting an unbridged metallocene complex according to the formula

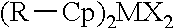

wherein, R is hydrogen or a hydrocarbyl group; Cp is a cyclopentadienyl group; M is Zirconium or Hafnium; and X is halogen; with a propylene composition in the presence of a modified methyl aluminum oxide activator compound and under suitable oligomerization conditions. In general, the use of the combination of a modified methyl aluminum oxide activator compound with an unbridged metallocene complex has been found to provide the benefits described herein.

The propylene oligomer made according to the method comprises oligomer according to formula (I)

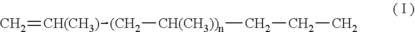

wherein, n=0 to about 1000.

While not limited thereto, in one embodiment, the modified methyl aluminum oxide activator compound may be of the formula [(CH₃)₍₁₋ₘ₎RₘAlO]ₙ, wherein 0.02<m<0.50, and R is a C₃ to C₁₅ linear or branched alkyl group.

### DETAILED DESCRIPTION

Although illustrative embodiments of one or more aspects are provided herein, the disclosed processes may be implemented using any number of techniques. The disclosure is not limited to the illustrative or specific embodiments, drawings, and techniques illustrated herein, including any exemplary designs and embodiments illustrated and described herein, and may be modified within the scope of the appended claims along with their full scope of equivalents.

Unless otherwise indicated, the following terms, terminology, and definitions are applicable to this disclosure. If a term is used in this disclosure but is not specifically defined herein, the definition from the IUPAC Compendium of Chemical Terminology, 2nd ed (1997), may be applied, provided that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein is to be understood to apply.

The term “Periodic Table” refers to the version of the IUPAC Periodic Table of the Elements dated Jun. 22, 2007, and the numbering scheme for the Periodic Table Groups is as described in Chem. Eng. News, 63(5), 26-27 (1985).

In this disclosure, while compositions and methods or processes are often described in terms of “comprising” various components or steps, the compositions and methods may also “consist essentially of” or “consist of” the various components or steps, unless stated otherwise.

The terms “a,” “an,” and “the” are intended to include plural alternatives, e.g., at least one. For instance, the disclosure of “a transition metal” or “an alkali metal” is meant to encompass one, or mixtures or combinations of more than one, transition metal or alkali metal, unless otherwise specified.

All numerical values within the detailed description and the claims herein are modified by “about” or “approximately” the indicated value, and account for experimental error and variations that would be expected by a person having ordinary skill in the art.

The term “olefin” refers to a hydrocarbon that has at least one carbon-carbon double bond that is not part of an aromatic ring or ring system. The term “olefin” includes aliphatic and aromatic, cyclic and acyclic, and/or linear and branched compounds having at least one carbon-carbon double bond that is not part of an aromatic ring or ring system, unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s). Depending on the context, the term “olefin” may refer to an “olefin oligomer” or an “olefin monomer.”

An “olefin oligomer” is an oligomer made from oligomerization of “olefin monomers.” For example, a “propylene oligomer” is made from the oligomerization of a propylene composition comprising propylene monomers. A product of the oligomerization process typically includes a mixture of branched olefin oligomers with a distribution of carbon numbers. Oligomer products resulting from oligomerization of monomers can be distilled or otherwise separated to further isolate or purify the olefin oligomer product to a preferred carbon range.

The term “alkyl” or related term refer to saturated hydrocarbon groups, which can be linear, branched, cyclic, or a combination of cyclic, linear and/or branched.

A “vinylidene-rich propylene oligomers” refers to propylene-based oligomers having a predominance of vinylidene moiety. An olefin oligomer having a vinylidene moiety is gem di-substituted at the internal end of the terminal double bond. Conventionally prepared propylene oligomers are typically rich in molecules with tri- or tetra-substituted internal double bond(s).

As used herein, the term “substituted” means that a hydrogen group has been replaced with substituent group, such as an alkyl group, an aromatic group, heteroatom, or a heteroatom-containing group.

The disclosure herein of combinations, subsets, groups, etc. of elements (e.g., combinations of components in a composition, or combinations of steps in a method) is intended to mean that all individual and collective combinations and permutations of such elements are included within the scope of the disclosure.

The propylene oligomers of the present invention are characterized by a highly regular structure and high vinylidene content, generally featuring a main chain with a long linear backbone and regularly spaced methyl groups. The high vinylidene propylene oligomers are long chain terminal olefins with a branch on every other carbon in the chain starting with the geminal branch on the vinylidene olefin. When numbered from the terminal olefin carbon the branches are positioned on even numbered carbons in the chain with the last three carbons the oligomer chain being unsubstituted or unbranched or deviate from the regular branching of the main oligomer chain in other ways.

In general, propylene oligomer comprises oligomer according to formula (I)

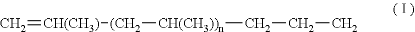

wherein, n is broadly in the range of from 0 to about 1000, or more specifically from 0 to about 800, or 700, or 600, or 500, or 400, or 300, or 200, or 100. In terms of number average molecular weight, the propylene oligomer typically has a number average molecular weight Mₙ is in the range of about 200, 250, 300, 400, 500, 600, 650, 700, or 750 to about 2000, 3000, 4000, 5000, 6000, 7000, 8000, or 10,000. Generally, the Mₙ of the propylene oligomer can be in a range from any minimum Mₙ disclosed herein to any maximum Mₙ disclosed herein.

In some embodiments, the high vinylidene propylene oligomers are products of oligomerization wherein at least 70 mol % of the propylene oligomers have a vinylidene moiety. In some embodiments, at least 80 mol %, 90 mol %, or 95 mol % of the propylene oligomers have a vinylidene moiety. Side products of the oligomerization may include oligomers that do not have vinylidene moiety. These oligomers may, in some cases, also comprise other moieties/configurations at the terminal double bond, such as tri-substituted, tetra-substituted, vinyl, and di-substituted (cis or trans).

While propylene is the main olefin monomer in the oligomerization reaction, the feed source can have a mixture of olefins having different numbers of carbon atoms, or olefins having predominantly a single number of carbon atoms. In some embodiments, the olefin feed may comprise at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, or at least 95 wt. % of propylene. The feed olefin(s) may further be introduced to the oligomerization reaction in a mixture with one or more non-olefinic hydrocarbons, such as alkanes or aromatics. In general, the propylene oligomer composition comprises propylene oligomer comprising from about 50 to 100 wt. % propylene and from 0 to about 50 wt. % of one or more comonomers, or from about 60 to 100 wt. % propylene and from 0 to about 40 wt. % of one or more comonomers, or from about 70 to 100 wt. % propylene and from 0 to about 30 wt. % of one or more comonomers, or from about 80 to 100 wt. % propylene and from 0 to about 20 wt. % of one or more comonomers. In some cases, the propylene oligomer may comprise at least 50 wt. %, 60 wt. %, 70 wt. %, 75 wt. %, 80 wt. %, 85 wt. %, 90 wt. %, or 95 wt. % C₁₂ to C₇₀ (e.g., C₁₂ to C₄₀, C₁₂ to C₃₀, C₁₂ to C₂₀, C₁₄ to C₇₀, C₁₄ to C₄₀, C₁₄ to C₃₀, C₁₄ to C₂₀, C₁₆ to C₇₀, C₁₆ to C₄₀, C₁₆ to C₃₀, C₁₆ to C₂₄, C₂₀ to C₇₀, C₂₀ to C₄₀, C₂₀ to C₃₀, or C₂₀ to C₂₄) oligomers. The oligomer product may also comprise less than 30 wt. %, 25 wt. %, 20 wt. %, 15 wt. %, 10 wt. %, 8 wt. %, 6 wt. %, 5 wt. %, 4 wt. %, 3 wt. %, or 2 wt. %>C₇₀ oligomers. The wt. % of the oligomer(s) disclosed herein is based upon the total weight of the propylene oligomer product.

The propylene oligomer further comprises less than about 5 wt. %, or less than about 4 wt. %, or less than about 3 wt. %, or less than about 2 wt. % , or less than about 1 wt. % of hydrogenated propylene oligomer.

While not limited thereto, the propylene monomer may be sourced from a petroleum cracking operation and used without separation of propylene from propane prior to use in the oligomerization reaction. Such cracking operation(s) may comprise a catalytic cracking process, such as Fluid Catalytic Cracking or a thermal cracking process, such as steam cracking or Coking. The cracking operation may also involve propane dehydrogenation.

The vinylidene propylene oligomers of the present invention possess a high degree of structural regularity and a high terminal vinylidene content and are prepared using suitable single site metallocene catalyst(s) that allow for the control of side chain length and/or branching and provide a high content of terminal vinylidene groups. By comparison with propylene oligomers prepared using metallocene catalysts according to different methods, the present invention unexpectedly provides the ability to introduce a high content of terminal vinylidene groups and to reduce the content of hydrogenated propylene oligomer.

A metallocene is a well-known organometallic molecule complex typically containing zirconium, titanium, hafnium, Group 4-6 transition metals, lanthanide metals, and the like. The metal typically sits at or near the center of the complex and coordinates to two cyclic alkyl anions, such as cyclopentadienyl groups. A more detailed discussion of metallocene catalysts may be found in U.S. Pat. Nos. 6,511,568; 8,536,391; and 9,732,300.

In general, useful metallocenes are unbridged complexes according to the formula

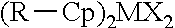

wherein, R is hydrogen or a hydrocarbyl group; Cp is a cyclopentadienyl group; M is Zirconium or Hafnium; and X is halogen;

The method of making the propylene oligomer as described herein generally involves contacting an unbridged metallocene complex according to the formula

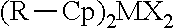

wherein, R is hydrogen or a hydrocarbyl group; Cp is a cyclopentadienyl group; M is Zirconium or Hafnium; and X is halogen; with a propylene composition in the presence of a modified methyl aluminum oxide (MMAO) activator compound and under oligomerization conditions; to form the propylene oligomer; wherein, the propylene oligomer comprises less than about 6 wt. % of hydrogenated propylene oligomer, a number average molecular weight Mₙ of about 200 to about 10,000, and a vinylidene content of greater than about 70 mol %. The oligomer product produced is a propylene oligomer (i.e., the repeating units of the olefin oligomer can be substantially all propylene units). In some cases, the repeating units of the oligomer can contain at least about 80 mol %, or at least about 90 mol %, at least 95 mol %, at least 98 mol %, or at least 99 mol % propylene units.

Suitable hydrocarbyl groups R comprise linear or branched, substituted or unsubstituted, saturated or unsaturated C₁ to C₃₀ hydrocarbyl group. Generally, the cyclopentadienyl group Cp is unsubstituted or substituted. Cyclopentadienyl groups known in the art may be used provided where such use is consistent with the invention described herein.

The modified methyl aluminum oxide (MMAO) activator compound is typically a liquid solution comprising the MMAO compound of the formula [(CH₃)₍₁₋ₘ₎RₘAlO]ₙ, wherein 0.02<m<0.50, and R is a C₃ to C₁₅ linear or branched alkyl group. Such compounds are commercially available from chemical suppliers, such as Sigma-Aldrich, Nouryon Functional Chemicals, and the like. While not limited thereto, useful modified methyl aluminoxane compounds include products identified and commercially available as MMAO-20, MMAO-12, MMAO-7, MMAO-21, MMAO-3A, and the like. Such compounds are provided as solvent solutions rather than as solids.

Suitable oligomerization conditions generally include any known in the art that are consistent with preparing the propylene oligomer described herein. For example, as described in U.S. Pat. No. 9,732,300, the oligomerization temperature can be in a range from 0° C. to 165° C. In some cases, the oligomerization temperature can be in a range from 20° C. to 160° C., from 40° C. to 160° C., or from 40° C. to 150° C., while in other cases, the oligomerization temperature can be in a range from 50° C. to 150° C., from 50° C. to 140° C., or from 50° C. to 130° C. I still further cases, the oligomerization temperature can be in a range from 60° C. to 130° C., from 60° C. to 120° C., or from 60° C. to 90° C., or from 30° C. to 130° C., from 30° C. to 120° C., or from 30° C. to 100° C. Other appropriate oligomerization temperatures and temperature ranges are readily apparent from this disclosure.

Suitable reaction pressures (or propylene partial pressures) generally include any known in the art that are consistent with preparing the propylene oligomer described herein. For example, the reaction pressure (or propylene partial pressure) under which the oligomerization is conducted can be in a range from 50 psig (344 kPa) to 4,000 psig (27.6 MPa), from 100 psig (689 KPa) to 3,000 psig (20.9 MPa), or from 150 psig (1.0 MPa) to 2500 psig (17.2 MPa). In some cases, the reaction pressure (or propylene partial pressure) can be in a range from 200 psig (1.4 MPa) to 2500 psig (17.2 MPa), from 200 psig (1.4 MPa) to 2,000 psig (13.8 MPa), from 250 psig (1.4 MPa) to 2,000 psig (1.7 MPa), or from 250 psig (1.5 MPa) to 1,500 psig (10.3 MPa). Other appropriate reaction pressures (or propylene partial pressures) are readily apparent from this disclosure.

The oligomerization process may also be conducted in the presence of a certain amount of added hydrogen. In such cases, the oligomer product can be formed in the presence of hydrogen, i.e., the olefin feedstock (containing propylene), the catalyst system, and hydrogen can be contacted to form the oligomer product under oligomerization conditions. For instance, the oligomer product can be formed at a hydrogen partial pressure of at least 1 psig (6.9 kPa), 5 psig (34 kPa), 10 psig (69 kPa), 25 psig (172 kPa), or 50 psig (345 kPa); additionally or alternatively, the oligomer product can be formed at a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), or from 50 psig (345 kPa) to 500 psig (3.4 MPa). Other appropriate hydrogen partial pressures are readily apparent from this disclosure.

In other terms, the molar ratio of hydrogen to propylene may be generally 0.04 or less, or 0.03 or less, or 0.02 or less or 0.01 or less. In some cases, the present invention provides the ability to reduce the degree of hydrogenated propylene oligomer product produced while still maintaining a high degree of propylene conversion to propylene oligomer product as compared with other metallocene/activator systems used to prepare propylene oligomers. For example, the present invention allows for high propylene conversion (i.e., greater than 90% or 95%) at a hydrogen:propylene molar ratio of 0.01 while producing a low amount of hydrogenated propylene oligomer product (i.e., less than 5 wt. %, or in the range of 2-3.5 wt. %). By comparison, the solid activator/metallocene catalyst system of U.S. Pat. No. 9,723,300 produced a propylene oligomer product having a substantially greater content of hydrogenated propylene oligomer (i.e., about 9 wt. %) at a propylene conversion of only about 50%.

### EXAMPLES

The following examples provide representative embodiments for the preparation of propylene oligomers according to the invention and the preparation of comparative propylene oligomers. The vinylidene content was determined using ¹H NMR spectroscopy by comparing the integral of the resonances in the vinylidene R,R′C═CH₂ range 4.5-4.8 ppm with integral of other olefins in the C═C—H range 4.5-5.9 ppm correcting for the number of protons each resonance represents. The respective olefins are identified as follows:
4.5-4.8 ppm: R,R′C═CH2 (two vinylidene protons)
4.8-5.2 ppm: RR′C═CHR″ (one trisubstituted double bond proton) and R—CH═CH₂ (two vinyl protons)
5.2-5.6 ppm: R—CH═CHR′ (two protons on disubstituted internal double bond)
5.6-5.9 ppm: R—CH═CH₂: (one proton on internal carbon in vinyl group)

### Example 1—Reference Example According to U.S. Pat. No. 9,732,300

### Propylene Oligomerization Using Triisobutyaluminum (TIBA) in Combination With Fluorinated Silica-Alumina SSA-1

Fluorinated silica-alumina, SSA-1, was prepared as described in U.S. Pat. No. 9,732,300 (column 28 line 65 to column 29 line 12) by treatment of calcined alumina with tetraethylorthosilicate, calcination and subsequent fluorination of the thus formed silica coated alumina by treatment with ammonium bifluoride solution to incipient wetness and calcining in dry air at 600° C.

1.0 gram of the SSA-1 powder slurried in dry anhydrous heptane, 0.56 ml 1 molar TIBA in hexane and 4.5 ml of a 0.006 molar solution of bis(tert-butylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 215 g propylene oligomer. The vinylidene content in the oligomers was determined by ¹H NMR spectroscopy to be approx. 35 mol %.

### Example 2—Reference Example According to U.S. Pat. No. 8,536,391

### Propylene Oligomerization Using Triisobutyaluminum (TIBA) in Combination With Fluorinated Silica-Alumina SSA-2

Fluorinated silica-alumina, SSA-2, was prepared as described in U.S. Pat. No. 8,536,391 (example 1) by treatment of silica-alumina with aqueous ammonium bifluoride to incipient wetness, drying and calcining the product in dry air at 400° C.

1.0 gram of the SSA-2 powder slurried in dry anhydrous heptane, 0.56 ml 1 molar TIBA in hexane and 4.5 ml of a 0.006 molar solution of bis(tert-butylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 189 g propylene oligomer. The vinylidene content in the oligomers was determined by ¹H NMR spectroscopy to be approx. 21 mol %.

### Example 3—Example According to Invention

MMAO-20 is a commercially available product containing isobutyl modified methyl aluminum oxide in hexane solution in a concentration corresponding to 7 wt. % Al or approximately 2.3 mole aluminum per liter

1.65 ml 2.3 molar MMAO-20 solution and 4.5 ml of a 0.006 molar solution of bis(tert-butylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 237 g propylene oligomer product. The vinylidene content in the oligomers was determined by ¹H NMR spectroscopy to be approx. 91%.

### Example 4—Example According to Invention

1.65 ml 2.3 molar MMAO-20 solution and 4.5 ml of a 0.006 molar solution of bis-(isopropylcyclopentadienyl) zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 237 g propylene oligomer. The vinylidene content in the oligomers was determined by ¹H NMR spectroscopy to be approx. 82 mol %.

### Example 5—Example According to Invention

1.65 ml 2.3 molar MMAO-20 solution and 4.5 ml of a 0.006 molar solution of bis(isopropylcyclopentadienyl)hafnium dichloride was loaded into a 1 liter stirred autoclave reactor together with 700 ml dry liquid mixture containing 70 wt. % propylene and 30% propane. 7.7 normal liter of hydrogen was also added, and the reactor heated to 70° C. After reaction overnight (about 18 hrs) the autoclave was depressurized, and the content was withdrawn and washed with water to yield 187 g propylene oligomer. The vinylidene content in the oligomers was determined by ¹H NMR spectroscopy to be approx. 84 mol %.

### Example 6—Example According to Invention

1.65 ml 2.3 molar MMAO-20 solution and 4.5 ml of a 0.006 molar solution of bis(n-butylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 200 g propylene oligomer. The vinylidene content in the oligomers was determined by ¹H NMR spectroscopy to be approx. 94 mol %.

### Example 7—Example According to Invention

1.65 ml 2.3 molar MMAO-20 solution and 4.5 ml of a 0.006 molar solution of bis(ethylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 230 g propylene oligomer. The vinylidene content in the oligomers was determined by ¹H NMR spectroscopy to be approx. 90 mol %.

### Example 8—Example According to Invention

1.65 ml 2.3 molar MMAO-20 solution and 4.5 ml of a 0.006 molar solution of bis(methylcyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 220 g propylene oligomer. The vinylidene content in the oligomers was determined by ¹H NMR spectroscopy to be approx. 89 mol %.

### Example 9—Example According to Invention

1.65 ml 2.3 molar MMAO-20 solution and 4.5 ml of a 0.006 molar solution of bis(cyclopentadienyl)zirconium dichloride was loaded into a 1 liter stirred autoclave reactor together with 700 ml dry liquid mixture containing 70 wt. % propylene and 30 wt. % propane. 7.7 normal liter of hydrogen was also added, and the reactor heated to 70° C. After reaction overnight (about 18 hrs) the autoclave was depressurized, and the content was withdrawn and washed with water to yield 201 g propylene oligomer. The vinylidene content in the oligomers was determined by ¹H NMR spectroscopy to be approx. 87 mol %.

The foregoing description of one or more embodiments of the invention is primarily for illustrative purposes, it being recognized that variations might be used which would still incorporate the essence of the invention. It will be understood that the invention is not limited to the embodiments described above and various modifications and improvements can be made without departing from the concepts described herein. Except where mutually exclusive, any of the features may be employed separately or in combination with any other features and the disclosure extends to and includes all combinations and sub-combinations of one or more features described herein. Reference should be made to the following claims in determining the scope of the invention.

For the purposes of U.S. patent practice, and in other patent offices where permitted, all patents and publications cited in the foregoing description of the invention are incorporated herein by reference to the extent that any information contained therein is consistent with and/or supplements the foregoing disclosure.

1. A propylene oligomer composition comprising a propylene oligomer having a number average molecular weight Mₙ of about 200 to about 10,000 and a vinylidene content of greater than about 70 mol %; wherein, the propylene oligomer composition comprises less than about 6 wt. % of hydrogenated propylene oligomer and from about 70 wt. % to 100 wt. % of the propylene oligomer.

2. The propylene oligomer composition of claim 1, wherein the propylene oligomer comprises oligomer according to formula (I) 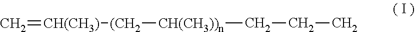
wherein, n=0 to about 1000.

3. The propylene oligomer composition of claim 1, wherein the vinylidene content of the propylene oligomer is greater than about 80 mol %.

4. The propylene oligomer composition of claim 1, wherein the number average molecular weight Mₙ is in the range of about 250, to about 10,000.

5. The propylene oligomer composition of claim 1, wherein the propylene oligomer composition comprises less than about 5 wt. % of hydrogenated propylene oligomer.

6. The propylene oligomer composition of claim 1, wherein the propylene oligomer composition comprises propylene oligomer comprising from about 50 to 100 wt. % propylene and from 0 to about 50 wt. % of one or more comonomers.

7. A propylene oligomer according to formula (I) 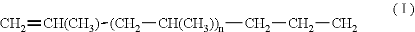
wherein, n=0 to about 1000; the propylene oligomer having a number average molecular weight Mₙ of about 200 to about 10,000 and a vinylidene content of greater than about 70 mol %.

8. The propylene oligomer of claim 7, wherein the vinylidene content of the propylene oligomer is greater than about 80 mol %.

9. The propylene oligomer of claim 7, wherein the number average molecular weight Mₙ is in the range of about 250 to about 10,000.

10. The propylene oligomer of claim 7, wherein the propylene oligomer comprises less than about 5 wt. % of hydrogenated propylene oligomer.

11. A method of making a propylene oligomer having a number average molecular weight Mₙ of about 200 to about 10,000 and a vinylidene content of greater than about 70 mol %; the method comprising
contacting an unbridged metallocene complex according to the formula 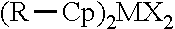
wherein, R is hydrogen or a hydrocarbyl group; Cp is a cyclopentadienyl group; M is Zirconium or Hafnium; and X is halogen;
with a propylene composition in the presence of a modified methyl aluminum oxide activator compound and under oligomerization conditions;
to form the propylene oligomer; wherein, the propylene oligomer comprises less than about 6 wt. % of hydrogenated propylene oligomer.

12. The method of claim 11, wherein the propylene oligomer comprises oligomer according to formula (I) 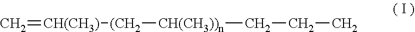
wherein, n=0 to about 1000.

13. The method of claim 11, wherein the hydrocarbyl group R comprises a linear or branched, substituted or unsubstituted, saturated or unsaturated C₁ to C₃₀ hydrocarbyl group.

14. The method of claim 11, wherein the cyclopentadienyl group Cp is unsubstituted or substituted.

15. The method of claim 11, wherein the modified methyl aluminum oxide activator compound is of the formula [(CH₃)₍₁₋ₘ₎RₘAlO]ₙ, wherein 0.02<m<0.50, and R is a C₃ to C₁₅ linear or branched alkyl group.

16. The method of claim 11, wherein the vinylidene content of the propylene oligomer is greater than about 80 mol %.

17. The method of claim 11, wherein the number average molecular weight Mₙ is in the range of about 250 to about 10,000.

18. The method of claim 11, wherein the propylene composition comprises from about 50 to 100 wt. % propylene and from 0 to about 50 wt. % of one or more comonomers.

19-21. (canceled)
